(19) United States
(12) Patent Application Publication
(10) Pub. No.: US 2025/0222348 A1
(43) Pub. Date: Jul. 10, 2025

(54) WEARABLE HAPTIC DEVICE

(71) Applicant: MAS INNOVATION (PRIVATE) LIMITED, Battaramulla (LK)

(72) Inventor: Ross D. Alcazar, Aurora, IL (US)

(73) Assignee: MAS INNOVATION (PRIVATE) LIMITED, Battaramulla (LK)

(21) Appl. No.: 18/682,724

(22) PCT Filed: Sep. 8, 2023
(86) PCT No.: PCT/SG2023/050608

§ 371 Date: Feb. 9, 2024

(30) Foreign Application Priority Data
Sep. 30, 2022 (SG) 10202251224W

(51) Int. Cl. A63F 13/285; A41D 1/00; A61F 13/10; A61H 23/02; A61M 21/00; A61M 21/02; G06F 3/01

(52) U.S. Cl. CPC A63F 13/285; A41D 1/002; A61F 13/10; A61H 23/0218; A61H 23/0245; A61H 23/0263; A61M 21/02; G06F 3/014; G06F 3/016; A61H 2201/02; A61H 2201/1635; A61H 2201/165; A61H 2205/06; A61M 2021/0022; A61M 2021/0066; A63F 2300/1037

(57) ABSTRACT

A wearable haptic device includes: a garment for wearing on a user; a set of electronic elements attached to the garment. The electronic elements include a set of haptic elements; a control receptacle attached to the garment and communicatively connected to the electronic elements; and a control module removably connectable to the control receptacle, the control module configured for controlling the haptic elements, said controlling by the control module comprising generating haptic feedback from the haptic elements to the user. The control module is removable from the control receptacle and replaceable with another control module.

### CROSS REFERENCE TO RELATED APPLICATION(S)

The present disclosure claims the benefit of Singapore Patent Application No. 10202251224W filed on 30 Sep. 2022, which is incorporated in its entirety by reference herein.

### TECHNICAL FIELD

The present disclosure generally relates to a wearable haptic device.

### BACKGROUND

Many types of specialized garments have been developed for various applications, such as gaming or therapeutic applications. For example, compression garments can be worn to support muscle recovery. Some compression garments are integrated with massage functions to improve muscle recovery. Some of these compression garments may have different pre-programmed massage functions to suit the needs of different users, but the compression garments are ultimately still limited to their massage or therapeutic applications.

Therefore, in order to address some of these challenges, there is a need to provide an improved wearable haptic device.

### SUMMARY

According to an aspect of the present disclosure, there is a wearable haptic device comprising: a garment for wearing on a user; a set of electronic elements attached to the garment, the electronic elements comprising a set of haptic elements; a control receptacle attached to the garment and communicatively connected to the electronic elements; and a control module removably connectable to the control receptacle, the control module configured for controlling the electronic elements, said controlling by the control module comprising generating haptic feedback from the haptic elements to the user, wherein the control module is removable from the control receptacle and replaceable with another control module.

A wearable haptic device according to the present disclosure is thus disclosed herein. Various features, aspects, and advantages of the present disclosure will become more apparent from the following detailed description of the embodiments of the present disclosure, by way of non-limiting examples only, along with the accompanying drawings.

### BRIEF DESCRIPTION OF THE DRAWINGS

FIGS. 1A and 1B are illustrations of a wearable haptic device according to embodiments of the present disclosure.

FIG. 2 is an illustration of the wearable haptic device worn on a user.

FIG. 3 is a block diagram illustration of various components of the wearable haptic device.

### DETAILED DESCRIPTION

For purposes of brevity and clarity, descriptions of embodiments of the present disclosure are directed to a wearable haptic device in accordance with the drawings. While aspects of the present disclosure will be described in conjunction with the embodiments provided herein, it will be understood that they are not intended to limit the present disclosure to these embodiments. On the contrary, the present disclosure is intended to cover alternatives, modifications and equivalents to the embodiments described herein, which are included within the scope of the present disclosure as defined by the appended claims. Furthermore, in the following detailed description, specific details are set forth in order to provide a thorough understanding of the present disclosure. However, it will be recognized by an individual having ordinary skill in the art, i.e. a skilled person, that the present disclosure may be practiced without specific details, and/or with multiple details arising from combinations of aspects of particular embodiments. In a number of instances, well-known systems, methods, procedures, and components have not been described in detail so as to not unnecessarily obscure aspects of the embodiments of the present disclosure.

In embodiments of the present disclosure, depiction of a given element or consideration or use of a particular element number in a particular figure or a reference thereto in corresponding descriptive material can encompass the same, an equivalent, or an analogous element or element number identified in another figure or descriptive material associated therewith.

References to “an embodiment/example”, “another embodiment/example”, “some embodiments/examples”, “some other embodiments/examples”, and so on, indicate that the embodiment(s)/example(s) so described may include a particular feature, structure, characteristic, property, element, or limitation, but that not every embodiment/example necessarily includes that particular feature, structure, characteristic, property, element or limitation. Furthermore, repeated use of the phrase “in an embodiment/example” or “in another embodiment/example” does not necessarily refer to the same embodiment/example.

The terms “comprising”, “including”, “having”, and the like do not exclude the presence of other features/elements/steps than those listed in an embodiment. Recitation of certain features/elements/steps in mutually different embodiments does not indicate that a combination of these features/elements/steps cannot be used in an embodiment.

As used herein, the terms “a” and “an” are defined as one or more than one. The use of “/” in a figure or associated text is understood to mean “and/or” unless otherwise indicated. The term “set” is defined as a non-empty finite organization of elements that mathematically exhibits a cardinality of at least one (e.g. a set as defined herein can correspond to a unit, singlet, or single-element set, or a multiple-element set), in accordance with known mathematical definitions. The recitation of a particular numerical value or value range herein is understood to include or be a recitation of an approximate numerical value or value range. The terms “first”, “second”, etc. are used merely as labels or identifiers and are not intended to impose numerical requirements on their associated terms.

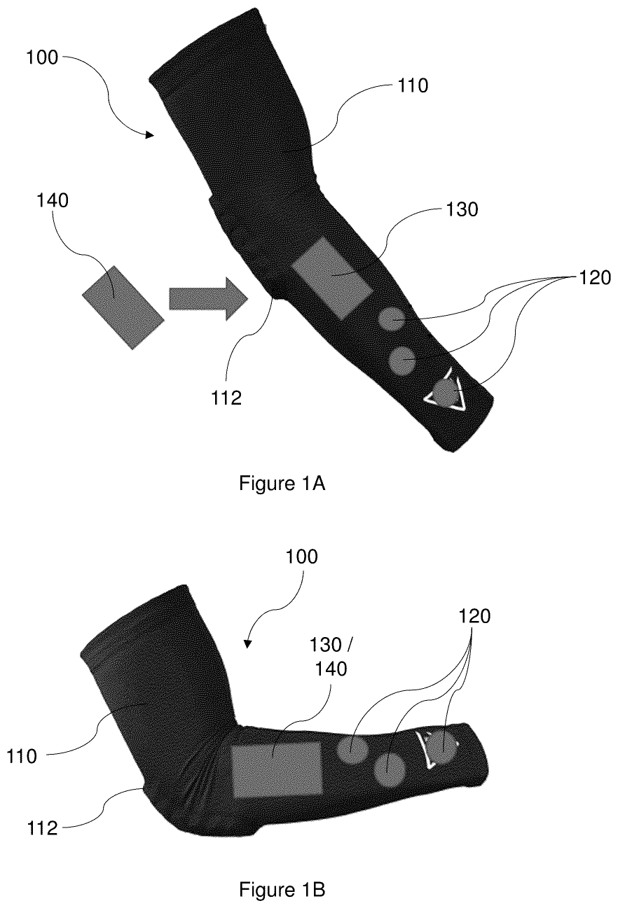

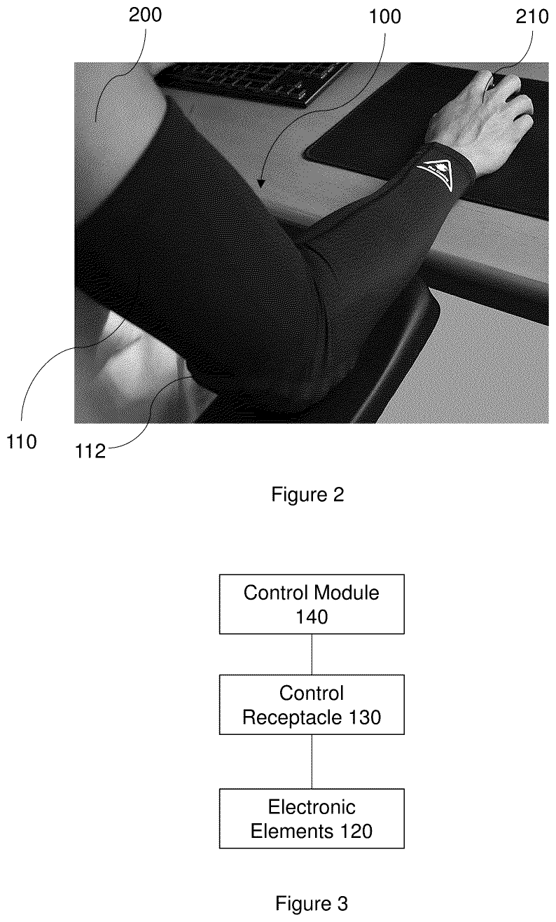

In representative or exemplary embodiments of the present disclosure, there is a wearable haptic device 100 as shown in FIGS. 1A and 1B. The wearable haptic device 100 is configured to provide haptic feedback to the user wearing the haptic device 100, wherein this haptic feedback uses physical stimuli to simulate tactile experiences for the user. FIG. 2 shows an example of the user wearing the haptic device 100 on the user's arm 200.

The wearable haptic device 100 includes a garment 110 for wearing on the user. In some embodiments, the garment 110 is in the form of a sleeve wearable on the user's arm 200 or leg. The garment 110 may be made of various fabric materials, such as spandex or nylon. The fabric material may optionally be stretchable. For example, the garment 110 includes a compression sleeve that may be made of a suitable compression fabric material. The compression sleeve is designed to compress against the user's arm 200 or leg, such as to aid muscle recovery. The compression sleeve may include fasteners such as zippers or touch fasteners to enhance the compression. The compression sleeve may include an elbow pad 112 for additional comfort to the user and to protect the user's elbow against impacts.

In some embodiments, the garment 110 may further include a glove wearable on the user's hand 210, wherein the glove is joined to the compression sleeve. The glove may provide partial or complete coverage of the hand 210. The glove may be removably joined to the compression sleeve, such as via fasteners like zippers or touch fasteners, so that the user can remove and replace either the compression sleeve or glove. Alternatively, the glove is permanently attached to the compression sleeve, such as by stitching or adhesive bonding. It will be appreciated that the garment 110 may be designed to be worn on other parts of the user's body, such as the leg, feet, head, neck, torso, or waist.

The wearable haptic device 100 includes a set of electronic elements 120 attached to the garment 110. The electronic elements 120 include a set of haptic elements that are configured to generate haptic or tactile feedback, such as vibrations, to the user based on input control signals to the haptic elements. In many embodiments, the haptic elements include mechanical actuators or vibration motors. In one embodiment, the haptic elements include eccentric rotating mass actuators. An eccentric rotating mass actuator is a vibration motor with an electrical motor that rotates an off-centred mass at high frequencies. In one embodiment, the haptic elements include linear resonant actuators. A linear resonant actuator is a vibration motor that produces an oscillating force across a single axis. In one embodiment, the haptic elements include piezoelectric actuators. A piezoelectric actuator operates based on the change in shape of a piezoelectric material when an electric field is applied. In one embodiment, the haptic elements include voice coil actuators. A voice coil actuator is a type of direct drive linear motor that converts electrical signals directly into linear magnetic force. It will be appreciated that the haptic elements may include any combination of, but are not limited to, the above examples.

The wearable haptic device 100 includes a control receptacle 130 attached to the garment 110 and communicatively connected to the electronic elements 120. The wearable haptic device 100 includes a control module 140 removably connectable to the control receptacle 130, the control module 140 configured for controlling the electronic elements 120, as shown in FIG. 3. Specifically, the control module 140, when connected to the control receptacle 130, is able to control the haptic elements to generate haptic feedback from the haptic elements to the user. The electronic elements 120 may include other components such as lighting elements and thermal elements that are similarly controllable by the control module 140. The control receptacle 130 may include electrical/electronic components that connect to corresponding components of the control module 140. The control receptacle 130 may include suitable seals to cover these electrical/electronic components when the control module 140 is not connected, such as to waterproof the electrical/electronic components.

In some embodiments, the electronic elements 120 include a set of lighting elements, wherein the lighting elements are controllable by the control module 140 to emit light from the lighting elements. For example, the lighting elements include LEDs. In some embodiments, the electronic elements 120 include a set of thermal elements, wherein the thermal elements are controllable by the control module 140 for heating or cooling. The thermal elements may thus be configured to provide heating or cooling effects to the user wearing the garment 110. For example, the thermal elements include conductive wires that generate heat in response to an electrical current flowing through them. In some embodiments, the electronic elements 120 include the haptic elements, the lighting elements, and the thermal elements.

In some embodiments, the wearable haptic device 100 includes a battery attached to the garment 110 for powering the electronic elements 120. The battery may be removable from the garment 110 to be replaced. Additionally or alternatively, the control module 140 includes a battery for powering the electronic elements 120. Specifically, when the control module 140 is connected to the control receptacle 130, the battery in the control module 140 powers the electronic elements 120. Optionally, the battery in the control module 140 complements the existing battery attached to the garment 110 to increase the overall battery capacity of the wearable haptic device 100.

When the control module 140 is connected to the control receptacle 130, the control module 140 becomes communicatively connected to the electronic elements 120. The user would be able to use the control module 140 to control the electronic elements 120. For example, the control module 140 may be used to control the haptic elements to generate vibrations for massage therapy. For example, the control module 140 may be used to control the lighting and thermal elements to generate various lighting and heating effects.

More particularly, the control module 140 is removably connected to the control receptacle 130 and the control module 140 can be removed and replaced or swapped with another control module 140. Different control modules 140 are configured for different applications and provide different functionalities. For example as mentioned above, the control module 140 may be configured for massage for muscle recovery, but other control modules 140 may be configured for other applications or functionalities.

Each control module 140 includes circuitry, such as a printed circuit board, and is configured with suitable control logic and software to control the other components of the wearable haptic device 100, including the electronic elements 120. More specifically, the control module 140 includes one or more processors having suitable logic, circuitry, and/or interfaces to execute instructions, codes, computer programs, and/or scripts. Some non-limiting examples of the processor include an application-specific integrated circuit (ASIC) processor, a reduced instruction set computing (RISC) processor, a complex instruction set computing (CISC) processor, a field-programmable gate array (FPGA), and the like. While only one processor is shown, multiple processors may be present. Thus, while instructions may be discussed as executed by a processor, the instructions may be executed simultaneously, serially, or otherwise executed by one or multiple processors (e.g. in a multi-core configuration).

In some embodiments, a first control module 140 is configured for health and wellness applications. The health and wellness applications may include massage therapy, thermal therapy, and/or sleep therapy. For example, the first control module 140 is configured to control the haptic elements to generate vibrations to provide massage therapy to the user. For example, the user wears the compression sleeve on the arm 200 and the vibrations may complement the compression effect from the compression sleeve for more holistic massage therapy, such as for muscle recovery. The haptic elements may be arranged such that the vibrations target certain muscles or muscle groups of the arm 200. For example, the first control module 140 is configured to control the thermal elements to generate heating effects for thermal therapy, such as to improve blood circulation. The thermal therapy may be combined with the massage therapy to facilitate muscle recovery. For example, the first control module 140 is configured to control the haptic elements and thermal elements for sleep therapy, such as to generate heating effects that improve blood circulation and sleep quality.

In some embodiments, a second control module 140 is configured for gaming applications. For example, the user wears the garment 110 including the sleeve and glove on the arm 200 and hand 210 for wireless gaming applications. The user may perform hand gestures with the glove to control various game functions. In one embodiment, the second control module 140 is configured for tracking movement of the user. For example, the wearable haptic device 100 includes sensors that communicate measure the position and orientation of the user's arm 200 and hand 210. For example, the sensors include inertial measurement units and/or accelerometers. The second control module 140 may also be configured to control the haptic elements to generate haptic feedback and the lighting elements to generate lighting effects. More specifically, the second control module 140 may receive data from the gaming applications and generate suitable haptic feedback and lighting effects in response to events in the gaming applications. Alternatively, the lighting effects may simply be for decorative purposes.

To further illustrate an example of the gaming applications, a virtual tennis game is executed in a computer system and the user wears the haptic device 100 with the second control module 140 to play the game. The wearable haptic device 100 may communicate with the computer system via known communication protocols, including wireless communication protocols such as Wi-Fi or Bluetooth. The second control module 140 sends the position and orientation data to the computer system to compute the movement of the user's arm 200 and hand 210. The user may use the garment 110 to simulate swinging a tennis racket to hit a tennis ball. Upon ‘impact’ between the tennis racket and the tennis ball in the game, the computer system sends a signal to the second control module 140 which in turn controls the haptic elements to generate vibrational feedback. This vibrational feedback simulates the tactile experience of the user hitting a real tennis ball with a real tennis racket.

Multiple control modules 140 can thus be configured for various applications with different functionalities. The first control module 140 can be used by the user for health and wellness applications. The user may later remove the first control module 140 and replace it with the second control module 140, so the user can use the wearable haptic device 100 for gaming applications. The user can continue to wear the garment 110 while hot swapping the control modules 140 on the control receptacle 130 for different applications. The same wearable haptic device 100 can thus be used for a wide range of applications by interchanging the control modules 140 and attaching the desired control module 140 to the same garment 110. These applications are not limited to those described herein and can include other applications such as virtual reality and augmented reality applications.

In the foregoing detailed description, embodiments of the present disclosure in relation to a wearable haptic device is described with reference to the provided figures. The description of the various embodiments herein is not intended to call out or be limited only to specific or particular representations of the present disclosure, but merely to illustrate non-limiting examples of the present disclosure. The present disclosure serves to address at least one of the mentioned problems and issues associated with the prior art. Although only some embodiments of the present disclosure are disclosed herein, it will be apparent to a person having ordinary skill in the art in view of this disclosure that a variety of changes and/or modifications can be made to the disclosed embodiments without departing from the scope of the present disclosure. Therefore, the scope of the disclosure as well as the scope of the following claims is not limited to embodiments described herein.

1. A wearable haptic device comprising:
a garment for wearing on a user;
a set of electronic elements attached to the garment, the electronic elements comprising a set of haptic elements;
a control receptacle attached to the garment and communicatively connected to the electronic elements; and
a control module removably connectable to the control receptacle, the control module configured for controlling the electronic elements, said controlling by the control module comprising generating haptic feedback from the haptic elements to the user,
wherein the control module is removable from the control receptacle and replaceable with another control module,
wherein the control module is configured for gaming applications.

2. The wearable haptic device according to claim 1, wherein the control module is configured for therapeutic applications.

3. (canceled)

4. The wearable haptic device according to claim 3, wherein the control module is configured for tracking movement of the user.

5. The wearable haptic device according to claim 1, wherein the control module comprises a battery for powering the electronic elements.

6. The wearable haptic device according to claim 1, further comprising a battery attached to the garment for powering the electronic elements.

7. The wearable haptic device according to claim 1, wherein the electronic elements comprise a set of lighting elements, wherein said controlling by the control module comprises emitting light from the lighting elements.

8. The wearable haptic device according to claim , wherein the electronic elements comprise a set of thermal elements, wherein said controlling by the control module comprises heating or cooling using the thermal elements.

9. The wearable haptic device according to claim 1, wherein the garment comprises a compression sleeve for wearing on an arm of the user.

10. The wearable haptic device according to claim 9, wherein the garment comprises a glove for wearing on a hand of the user, wherein the glove is joined to the compression sleeve.
